(19) United States
(12) Patent Application Publication
(10) Pub. No.: US 2026/0263058 A1
(43) Pub. Date: Sep. 10, 2026

(54) BIOPSY NEEDLE DEVICE AND METHODS OF USE

(71) Applicant: Boston Scientific Scimed, Inc., Maple Grove, MN (US)

(72) Inventor: Austin Grant JOHNSON, Worcester, MA (US)

(73) Assignee: Boston Scientific Scimed, Inc., Maple Grove, MN (US)

(21) Appl. No.: 19/556,065

(22) Filed: Mar. 4, 2026

Related U.S. Application Data
(60) Provisional application No. 63/767,049, filed on Mar. 5, 2025.

(51) Int. Cl. A61B 10/04; A61B 10/02

(52) U.S. Cl. CPC A61B 10/04; A61B 2010/0208; A61B 10/0283; A61B 2010/045

(57) ABSTRACT

A medical device may comprise a shaft having an inner sheath and an outer sheath, and a handle. The handle may include a housing defining a channel and the housing may be longitudinally fixedly coupled to the outer sheath. The handle may also include an actuator including a proximal grip portion and a plunger extending distally from the grip portion. A distalmost end of the plunger may include a widened portion, and the plunger may be fixedly coupled to the inner sheath. The widened portion may be longitudinally movable within the channel. A distance between a first surface of the housing and a second surface of the housing may define a maximum distance by which the actuator is movable relative to the housing and a maximum distance by which the inner sheath is movable relative to the outer sheath.

### CROSS-REFERENCE TO RELATED APPLICATIONS

This application claims the benefit of priority to U.S. Provisional Application No. 63/767,049, filed on Mar. 5, 2025, which is incorporated by reference herein in its entirety.

### TECHNICAL FIELD

The disclosure relates to medical devices and related methods. More particularly, the disclosure relates to a biopsy needle device used for obtaining tissue samples during biopsy or other procedures, and methods for using the devices.

### BACKGROUND

Biopsy procedures are commonly used in medical diagnostics to obtain tissue samples from various body parts for examinations. These procedures often rely on medical devices, such as endoscopes or other medical scopes, to access and visualize a patient's anatomical lumen. For example, once an endoscope is positioned at the target site, a biopsy needle may be advanced through the working channel of an endoscope to the desired location for tissue collection. The device is then manipulated to assist in visualization and sampling. The scope of the invention is defined by the attached claims, which set forth the features and embodiments of the present invention.

### SUMMARY

This disclosure includes devices and methods that are used to acquire biopsy samples endoscopically. In aspects, the disclosed devices and methods pertain to fine needle biopsy (FNB) procedures. Each of the aspects disclosed herein may include one or more of the features described in connection with any of the other disclosed aspects. The examples described herein may have any of these features in any combination.

In an example, a medical device may comprise a shaft having an inner sheath, an outer sheath, and a handle. The handle may include a housing defining a channel and the housing may be longitudinally fixedly coupled to the outer sheath. The handle may also include an actuator. The actuator may include a proximal grip portion and a plunger extending distally from the grip portion. A distalmost end of the plunger may include a widened portion that has a larger width than more proximal portions of the plunger, and the plunger may be fixedly coupled to the inner sheath. The widened portion may be longitudinally movable within the channel. A distance between a first surface of the housing and a second surface of the housing may define a maximum distance by which the actuator is movable relative to the housing and a maximum distance by which the inner sheath is movable relative to the outer sheath.

Any examples described herein may have any of these features alone or in combination. A distal portion of the inner sheath may be coupled to a needle. The needle and the inner sheath may have a same diameter. The inner sheath may be more flexible than the needle. A portion of the plunger may be between the grip portion, and the widened portion may be narrower than the grip portion and the widened portion. The housing may include a first wall portion, and the wall portion may include the first surface. The housing may include a second wall portion, and the second wall portion may include the second surface. The second surface may be a distalmost surface of the second wall portion. In addition, a gap may be between the first wall portion and the second wall portion. In some aspects, the plunger may include a ridge or an O-ring. The ridge or the O-ring may be configured to inhibit distal movement of the plunger when the plunger is in a fully retracted configuration. The plunger may include a slot, and the housing may have a protrusion received within the slot. The protrusion and the slot may be configured to inhibit distal movement of the plunger when the plunger is in a fully retracted configuration. The proximal end of the outer sheath may also be covered by a connector, and a ridge portion of the connector may be covered by a strain relief.

In another example, a medical device may comprise a shaft having an inner sheath, an outer sheath, a needle fixedly coupled to a distal end of the inner sheath, and a handle. The handle may include a housing fixedly coupled to the outer sheath and an actuator. The actuator may include a proximal grip portion and a plunger extending distally from the grip portion. A distalmost end of the plunger may include a widened portion that has a larger width than a portion of the plunger between the widened portion and the grip portion. The plunger may be fixedly coupled to the inner sheath. The widened portion may be longitudinally movable within the housing to actuate the inner sheath relative to the outer sheath. According to some aspects, the housing may include a first wall portion including a first surface, and a second wall portion having a second surface. The first surface may be a distal bound on movement of the widened portion, and the second surface may be a proximal bound on movement of the widened portion.

In another example, a medical method may comprise advancing a shaft of a medical device to a target site within a subject’s body. The shaft may include an outer sheath and an inner sheath. The medical device may include a handle with a housing and an actuator. The actuator may include a plunger having a widened portion coupled to the inner sheath. The medical method may further comprise applying a distal force to the actuator to unlock a locking mechanism and moving the actuator distally relative to the housing. The widened portion may move distally within the housing to move the inner sheath distally relative to the outer sheath. A stroke length of the actuator may be defined by a distance between a first surface and a second surface of the housing. The widened portion may abut the first surface in a fully retracted configuration of the inner sheath and may abut the second surface in a fully extended configuration of the inner sheath. According to some aspects, the locking mechanism may include a ridge or an O-ring on the plunger. The locking mechanism may also include at least one longitudinal slot on the plunger.

### BRIEF DESCRIPTION OF THE DRAWINGS

The accompanying drawings, which are incorporated in and constitute a part of this specification, illustrate examples of this disclosure and, together with the description, explain the principles of the disclosure.

FIG. 1 illustrates a side view of a medical device according to aspects of this disclosure;

FIG. 2 illustrates a side view of a distal portion of the medical device of FIG. 1, according to aspects of this disclosure;

FIG. 3A illustrates a partial view of a proximal portion of the medical device of FIG. 1, according to aspects of the disclosure; and

FIGS. 3B-3F illustrate features of the proximal portion of FIG. 3A.

### DETAILED DESCRIPTION

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/- 10% of a stated value.

Reference is now made in detail to examples of this disclosure, aspects of which are illustrated in the accompanying drawings. Wherever possible, the same reference numbers will be used throughout the drawings to refer to the same or like parts. Unless otherwise specified, aspects of the disclosed embodiments may be combined in any suitable manner.

Aspects of the disclosure include a medical device configured to obtain tissue samples during biopsy or other medical procedures. In examples, the medical device may be used during a fine needle biopsy (FNB) procedure, although such a procedure is merely exemplary. Aspects of the disclosed devices may alternatively be used in, for example, fine needle aspiration (FNA) procedures.

The medical device includes a handle and a shaft. The shaft may include an outer sheath, an inner sheath, and a needle. The needle may be coupled to a distal portion of the inner sheath. For example, the needle may be disposed only at a distal end of the shaft, such that it does not extend all the way through the outer sheath. The handle of the device contains a housing and an actuator. The actuator may be coupled to the inner sheath. The actuator may be configured to move the inner sheath and the needle relative to the outer sheath and the housing. Specifically, the actuator’s proximal and distal movement facilitates the controlled advancement or retraction of the needle. Additionally, the device may incorporate a locking mechanism to secure the actuator in a fixed position. For example, when the actuator is fully retracted, the locking mechanism may help secure both the inner sheath and the needle, preventing unintended movement during delivery, prior to desired actuation to collect a biopsy sample.

Such embodiments of medical devices may improve the flexibility of a shaft of a medical device while maintaining adequate rigidity. For example, a more flexible inner sheath extends through most of the shaft, while the rigid needle is coupled to a distal portion of the inner sheath. The increased flexibility in portions of the shaft having the flexible inner sheath may help the shaft navigate tortuous body lumens or small-diameter passages without compromising the ability of the medical device to perform biopsy or other medical functions.

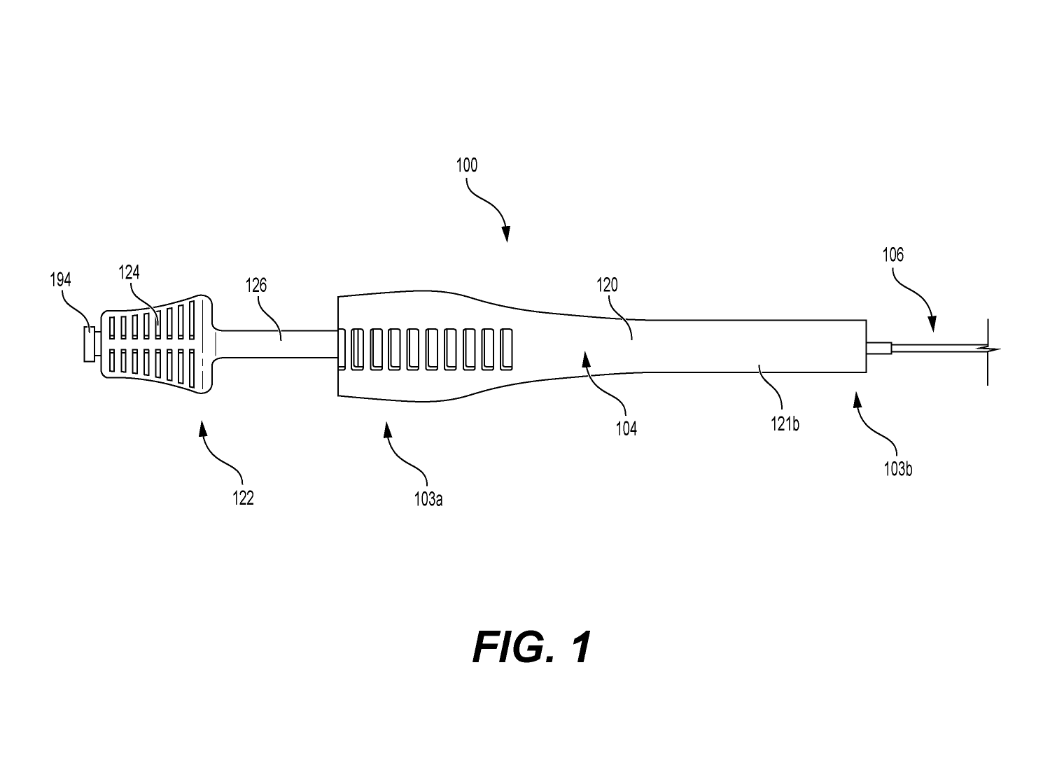

FIG. 1 illustrates a medical device 100, which includes a handle 104 and a shaft 106. Handle 104 may have a proximal portion 103a and a distal portion 103b. Proximal portion 103a of handle 104 may include an actuator 122, while shaft 106 may extend from distal portion 103b. Shaft 106 may be a tube extending from distal portion 103b of handle 104 and having a length sufficient to reach target sites within a subject's body. Additionally, shaft 106 is configured to be flexible to navigate tortuous anatomical pathways within various body lumens. For example, shaft 106 may be used with a bronchoscope to maneuver through the tortuous structures of the bronchial tree during lung biopsies or through the gastrointestinal tract in endoscopic procedures.

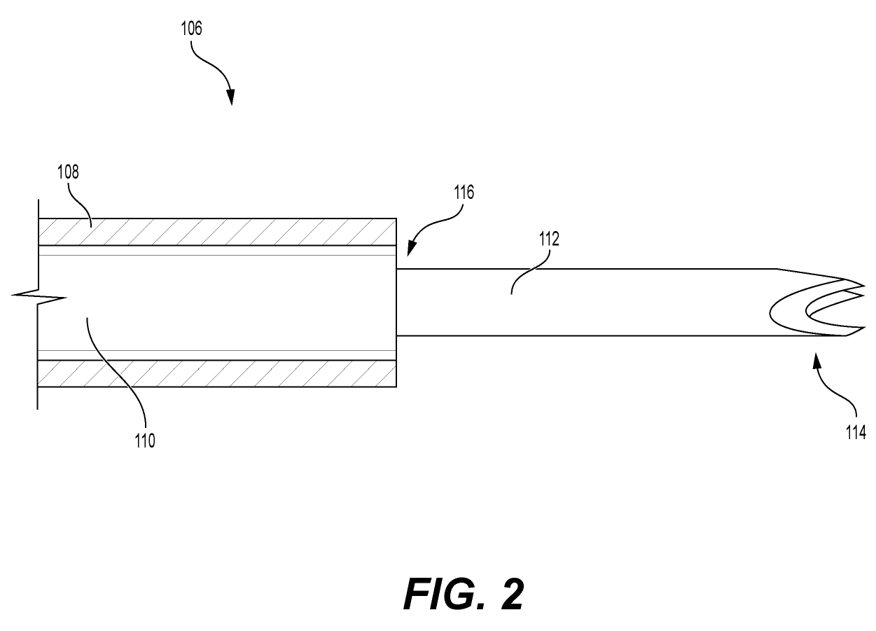

FIG. 2 shows a distal portion of shaft 106 including an outer sheath 108, an inner sheath 110, and a needle 112. Outer sheath 108 is shown in cross-section to reveal inner sheath 110. Outer sheath 108 and inner sheath 110 may be manufactured from materials like stainless steel coil, polytetrafluoroethylene (PTFE), braided polymer, Nitinol (nickel-titanium) tube (e.g., a hypotube), or combinations thereof. Outer sheath 108 may be formed from any material or by any process that provides outer sheath 108 with a desired flexibility and resilience profile. Outer sheath 108 may form the outermost layer of shaft 106. Alternatively, outer sheath 108 may be surrounded by an outer coating of material (not shown).

Outer sheath 108 may radially surround at least a portion of inner sheath 110. Inner sheath may be flexible. For example, inner sheath may be constructed from any of the materials listed above for outer sheath 108 (e.g., metal coils, polymers, plastics, etc.). In some aspects, inner sheath 110 and outer sheath 108 may have the same flexibility. In other aspects, one of inner sheath 110 or outer sheath 108 may be more flexible than the other.

Inner sheath 110 be coupled to needle 112 at a distal end of inner sheath 110. As described in further detail below, inner sheath 110 may be coupled to actuator 122 at a proximal end of inner sheath 110. As described below, actuator 122 may be manipulated by the user to move inner sheath 110 and needle 112 proximally or distally. For example, actuator 122 may be configured to transition shaft 106 from a retracted or proximal) configuration, in which a distalmost end of needle 112 is proximal of a distalmost end of outer sheath 108, to an extended (or distal) configuration, in which at least a portion of needle 112 is distal to a distalmost end of outer sheath 108. In other words, actuator 122 may transition shaft 106 from a sheathed configuration, in which needle 112 is covered by outer sheath 108, to an unsheathed configuration, in which needle 112 is uncovered by outer sheath 108 and able to collect tissue (e.g., biopsy) samples.

In embodiments, needle 112 does not extend the entire length of shaft 106. As noted, needle 112 may instead be coupled to the distal end (e.g., distalmost end) of inner sheath 110. Inner sheath 110 and needle 112 may have a same inner diameter, such that the junction or transition between the inner surfaces of needle 112 and inner sheath 110 are smooth. As a result, a stylet or other device (such as that discussed below) may not snag as it moves through an inner lumen of inner sheath 110 and into an inner lumen of needle 112. In aspects, inner sheath 110 and needle 112 may also have the same outer diameters at the junction where inner sheath 110 and needle 112 are coupled. This smooth junction may inhibit needle 112 or inner sheath 110 from snagging on an inner surface of outer sheath 108 as needle 112 and inner sheath 110 are actuated proximally or distally. Alternatively, as shown, needle 112 may have a smaller outer diameter than inner sheath 110. This smaller outer diameter of needle 112 may also inhibit any snagging on outer sheath 108. Manufacturing techniques such as heat shrink tubing, and the use of a mandrel may be used to maintain uniformity and ensure a consistent diameter at the junction.

Needle 112 may terminate in a pointed or sharp distal tip 114 to facilitate insertion into the target tissue. In some aspects, needle 112 may incorporate specialized geometries optimized for specific medical procedures, such as serrated edges for cutting or a curved shape to conform to the anatomy of particular tissues. Distal tip 114 may have any suitable shape (e.g., bevel tip, Franseen tip, Menghini tip, etc.). As discussed above, inner sheath 110 and needle 112 may include a lumen extending longitudinally through their central portions, serving various functions such as transmitting suction, providing a conduit for a stylet or other device, or sample collection.

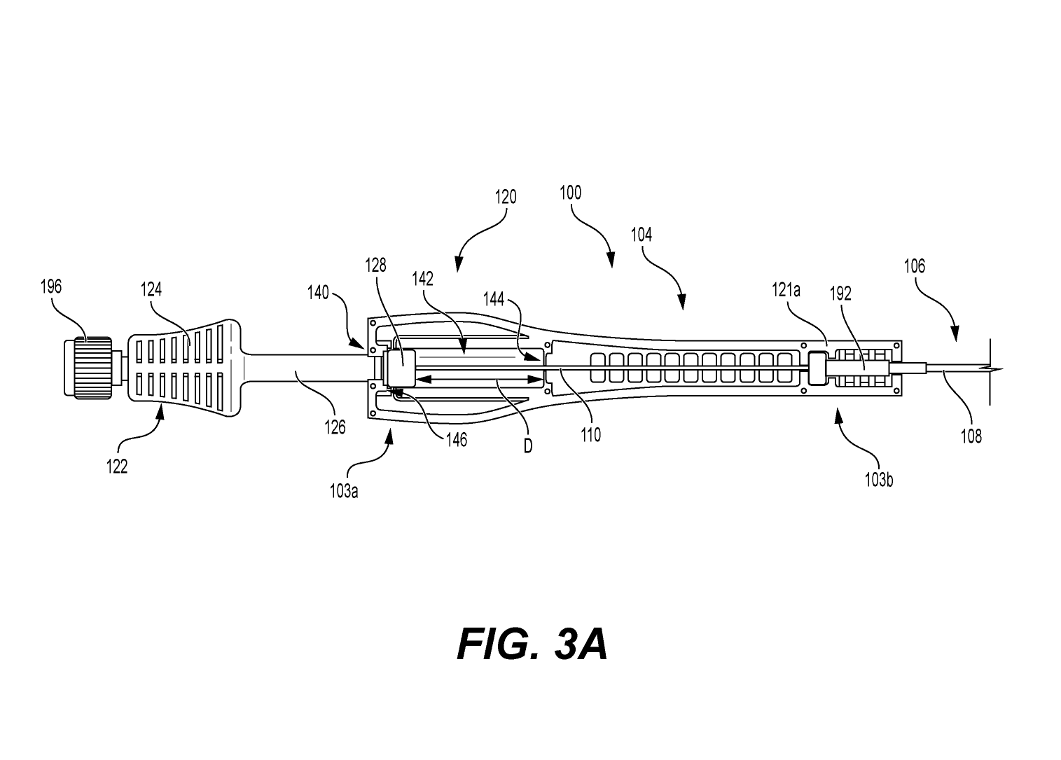

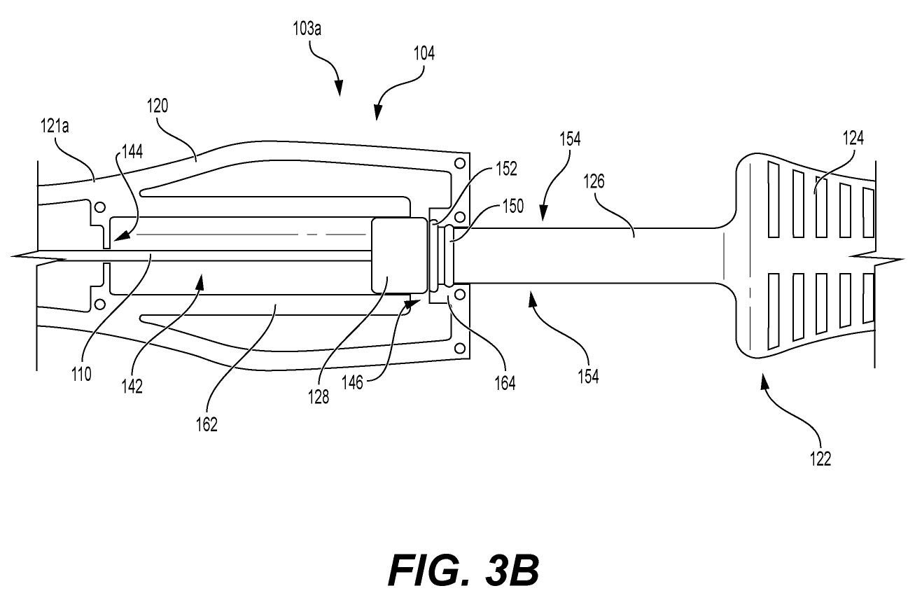

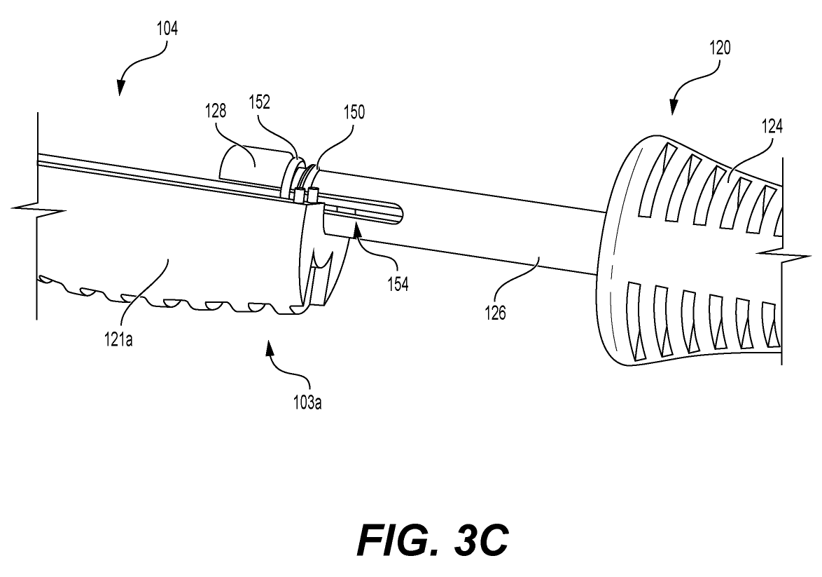

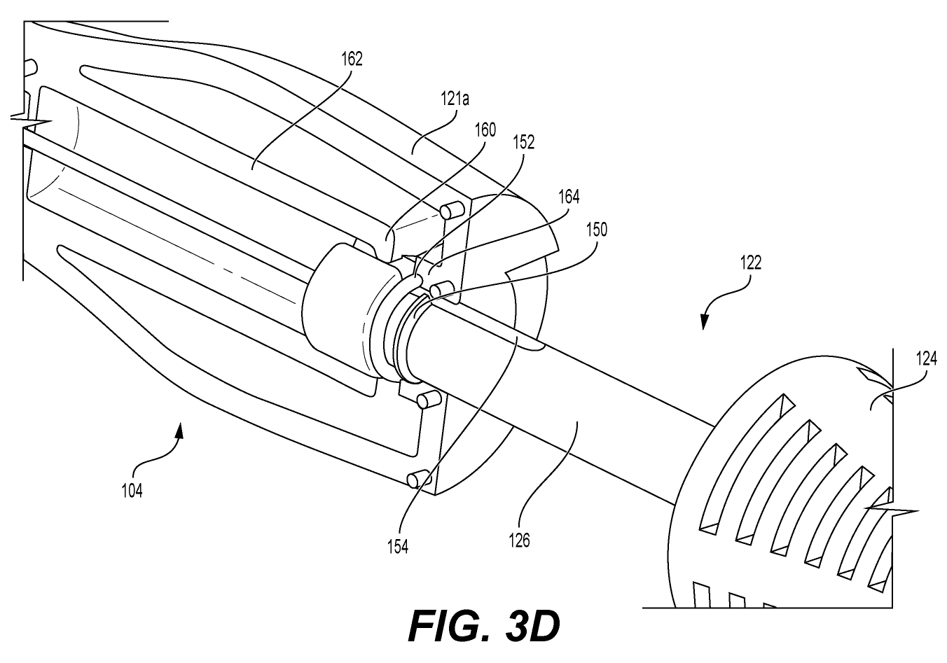

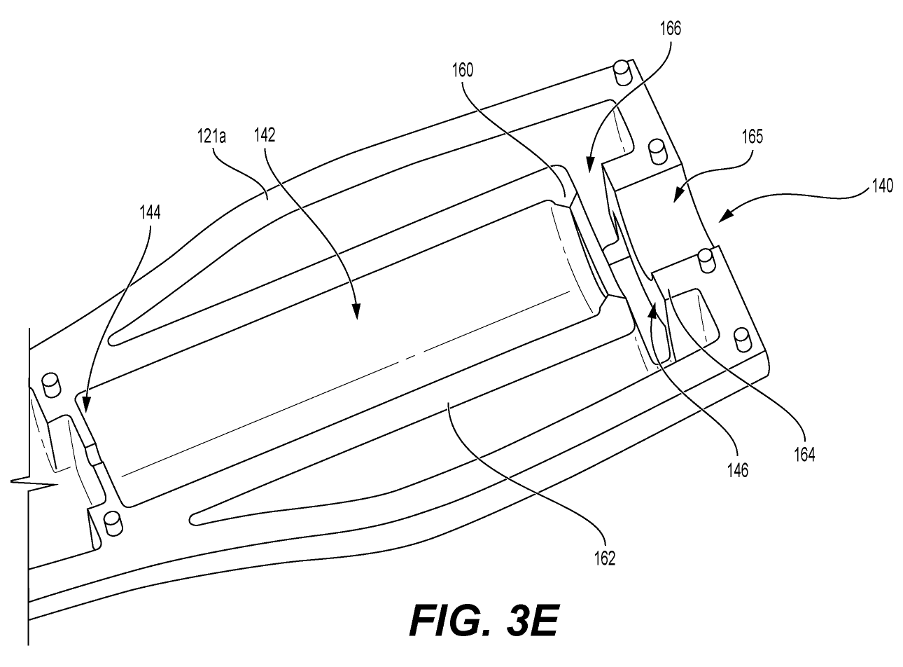

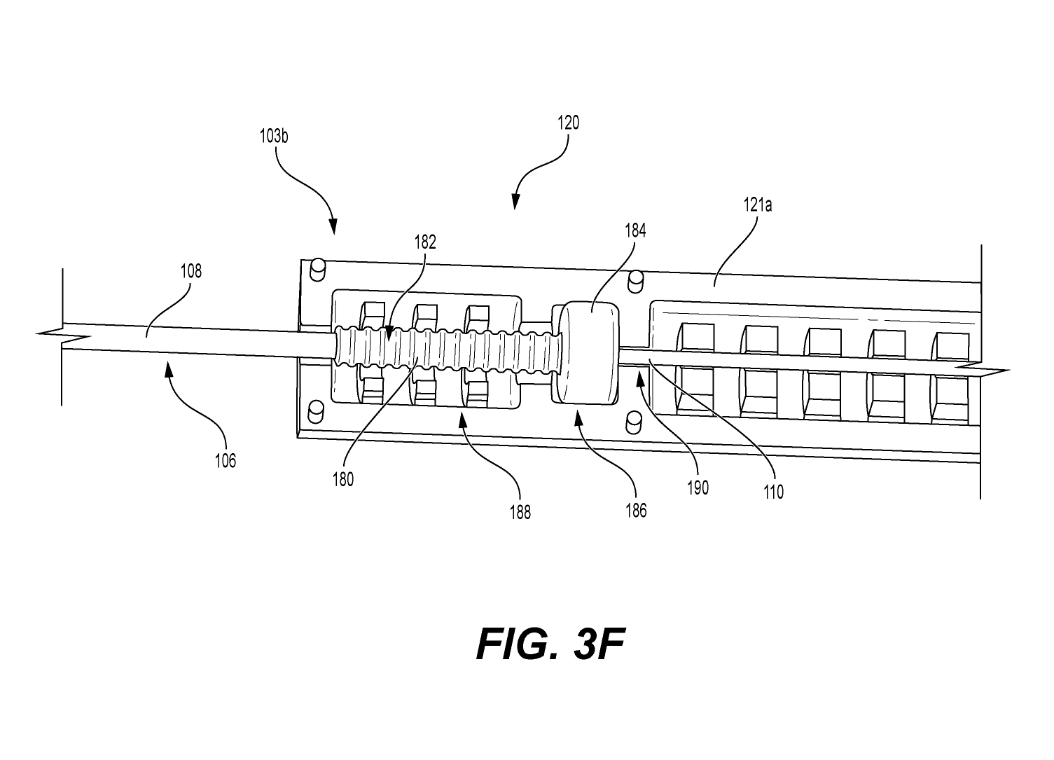

With reference to FIGS. 1 and 3A-3F, handle 104 may include a housing 120, which may consist of a first housing portion 121a (as shown in FIG. 3A) and a second housing portion 121b (as shown in FIG. 1). In FIGS. 3A-3D, second housing portion 121b has been removed to show internal features of handle 104. FIG. 3E shows first housing portion 121a in isolation. FIG. 3F, described in more detail below, shows handle 104 with second housing portion 121b removed and with other aspects also removed. In some aspects, first and second housing portions 121a and 121b may form two halves separated along a longitudinal plane at the center of housing 120. The first and second housing portions 121a and 121b may be fixed together using any suitable mechanism, such as by hinges, adhesive, friction fit, or other mechanisms. FIGS. 3B-3E shows housing 120 with second housing portion 121b removed, providing a clearer view of the internal components of housing 120.

A proximal portion of outer sheath 108 may extend into and be fixedly coupled to the distal portion 103b of housing 120, as described in further detail below. A proximal portion of inner sheath 110 may extend into housing 120, and a proximal end of inner sheath 110 may be coupled to actuator 122. Actuator 122 may move axially (longitudinally) relative to housing 120. Thus, movement of actuator 122 relative to housing 120 may move inner sheath 110 relative to outer sheath 108 and housing 120.

FIG. 3A also depicts a stylet 196. A distal end of stylet 196 may be inserted into a proximal lumen of actuator 122. Then, stylet may be extended through the lumen of inner sheath 110 and needle 112. Stylet 196 may have any properties of any stylet known in the art. Stylet 196 is omitted in, for example, FIG. 1 to show details of a proximal end of actuator 122.

As shown in FIGS. 3A and 3B, actuator 122 may include a grip 124 and a plunger 126. Grip 124 may be positioned at the proximal portion of actuator 122 and is designed for user manipulation. The distal portion of grip 124 may be wider or flared, while the proximal portion or base of grip 124 narrows slightly. The shape of grip 124 may be such that it provides for ergonomic grasping by a user’s fingers. Grip 124 may also include multiple grooves, ridges, or textured surfaces to help a user hold actuator 122.

A proximalmost end of grip 124 may be coupled to an adapter or connector 194. For example, connector 194 may be a Luer or other type of adapter. A source of suction/aspiration (e.g., a syringe) may be coupled to connector 194. Connector 194 may include an opening that is in fluid communication with the lumen of inner sheath 110/needle 112. Thus, fluid/aspiration delivered via connector 194 may be delivered through inner sheath 110 and needle 112. Stylet 196 may also be inserted into connector 194 and, in some examples, secured to actuator 122 using connector 194.

Plunger 126 may be integral with grip 124 (formed of a single piece of material). Plunger 126 may extend distally from grip 124 and through a proximal opening 140 in housing 120. As shown in FIGS. 3A-3D, plunger 126 may have a widened portion 128 at its distalmost end. Widened portion 128 may have a larger width (in a direction perpendicular to a longitudinal axis of plunger 126/handle 104) than more proximal portions of plunger 126. In some examples, widened portion 128 may be cylindrical, though other shapes, such as prism shapes, are also possible.

Housing 120 may define a channel 142, which may extend longitudinally through housing 120. Channel 142 may be configured to accommodate plunger 126, including widened portion 128. Opening 140 of housing 120 provides an entry point to channel 142, through which plunger 126 extends. Channel 142 may be an internal passage or groove formed within housing 120, providing a path for actuator 122 to move relative to housing 120.

As mentioned above, for ease of illustrating aspects of housing 120, FIG. 3E shows first housing portion 121a alone. Second housing portion 121b may be largely or wholly identical to first housing portion 121a. As shown particularly in FIG. 3E, first housing portion 121a may include a first wall portion 162. First wall portion 162 may define a portion of channel 142. The inner surface of first wall portion 162 may define outer (e.g., radially outer) sides of channel 142. First wall portion 162 may have a semi-circular cross-section, such that, when first housing portion 121a is coupled to second housing portion 121b, channel 142 has a cylindrical shape. In some aspects, first wall portion 162 may be contoured to match the outer dimensions of widened portion 128 to help guide the movement of widened portion 128 of plunger 126 and maintain longitudinal movement of widened portion 128 without excessive friction.

First housing portion 121a may also include a second wall portion 164 at the proximal end of first housing portion 121a. Second wall portions 164 of first housing portion 121a and may together define a channel 165. Channel 165 be shaped and dimensioned to receive a portion of plunger 126 that is proximal to widened portion 128. In other words, second wall portion 164 may be proximal to widened portion 128. In some aspects, contact between widened portion 128 and proximal surface 146 provides tactile feedback to the user, indicating that plunger 126 of actuator 122 has reached a fully retracted position.

As shown, particularly in FIGS. 3A, 3B, and 3E, a distal end of channel 142 of housing 120 may be defined by a distal surface 144. Distal surface 144 may be a distal surface of first wall portion 162. A proximal end of channel 142 may be defined by a proximal surface 146. Proximal surface 146 may be a distalmost surface of second wall portion 164. A distance between distal surface 144 and proximal surface 146 may define a maximum distance that actuator 122 may move within channel 142. Proximal surface 146 may define a proximal bound (or stop) on movement of widened portion 128. A proximal surface of widened portion 128 may abut proximal surface 146 of housing 120 when actuator 122 is in a proximalmost (fully retracted) configuration. Distal surface 144 may define a distal bound (or stop) on movement of widened portion 128. A distal surface of widened portion 128 may abut distal surface 144 when actuator is in a distalmost (fully extended) configuration. A distance D between the distal surface of widened portion 128 in the fully retracted configuration (as shown in FIG. 3A) and distal surface 144 may be a maximum distance along which actuator 122 may move. In other words, distance D may be a stroke length of needle 112.

Plunger 126 may include markings that indicate the extent of actuation of actuator 122. These markings may correspond to the extent of travel of inner sheath 110 and needle 112 relative to outer sheath 108, providing visual feedback to the user regarding the needle's position relative to housing 120. For example, plunger 126 may include markings along the length of plunger 126, with each mark corresponding to a specific position of needle 112 (e.g., a distance by which needle 112 extends out of a distal end of outer sheath 108). In some aspects, contact between widened portion 128 and proximal surface 146 provides tactile feedback to the user, indicating that plunger 126 of actuator 122 has reached a fully retracted position.

Handle 104 may also include locking mechanism for locking the actuator 122 and needle 112 in the fully retracted (unactuated) position. In aspects, one or more of the features discussed below may be omitted. The features disclosed below may be utilized in various combinations. As shown in FIGS. 3B-3D, plunger 126 may include a ridge 150, which extends around a distal portion of plunger 126. Ridge 150 may be a raised protrusion or lip on the exterior surface of plunger 126. The profile of ridge 150 may be curved or angled to facilitate engagement with corresponding features in housing 120. In some aspects, ridge 150 may extend circumferentially (annularly) around a portion of plunger 126.

Furthermore, plunger 126 may include an O-ring 152. In some aspects, plunger 126 may have only one of O-ring 152 or ridge 150. O-ring 152 may increase friction against the surfaces of housing 120, helping to maintain plunger 126 in a locked position. For example, the circular shape of O-ring 152 may contribute to uniform contact and pressure distribution around the circumference of plunger 126.

Ridge 150 and/or O-ring 152 may be received within channel 165 in the fully retracted configuration. Ridge 150 and/or O-ring 152 may have a slightly wider width than a width of channel 165, and a material of second wall portion 164 may be slightly deformable, to accommodate the increased width. In alternatives, ridge 150 and/or O-ring 152 may be compressible upon application of a sufficient force. In further alternatives, an outer diameter of ridge 150 and/or O-ring 152 may closely match a diameter of channel 165, such that there is a friction fit between an inner surface of second wall portion and ridge 150/O-ring 152.

As shown in FIG. 3E, housing 120 may include a gap 166 between first wall portion 162 and second wall portion 164. In some aspects, after overcoming a threshold distal force, plunger 126 may move distally and ridge 150 or O-ring 152 may move into gap 166, such that ridge 150 or O-ring 152 no longer provide resistance to distal movement of plunger 126. In alternatives, gap 166 may be omitted. Ridge 150 or O-ring 152 may have a smaller width/diameter than widened portion 128, such that ridge 150 or O-ring 152 may freely move once plunger 126 is moved distally an amount sufficient that ridge 150 or O-ring 152 is disposed within channel 142.

As shown, particularly in FIGS. 3C and 3D, plunger 126 may also include slots 154, which may be longitudinal openings. In aspects, plunger 126 may have two slots 154, which may be diametrically (180 degrees) opposed to one another. The depth and width of slot 154 may be tailored to interact with corresponding features in housing 120, as described below.

As shown in FIGS. 3D and FIG. 3E, housing 120, includes one or more protrusions 160 configured to engage with slots 154. Protrusion 160 may extend radially inward from a proximal end of first wall portion 162. For example, protrusion 160 may form a narrower collar of first wall portion 162. Protrusion 160 may extend circumferentially around an inner surface of first wall portion 162 or at discrete points on first wall portion 162 (e.g., at diametrically opposed points corresponding to positions of slots 154). Protrusion 160 may fit into the corresponding slot 154 on plunger 126. As plunger 126 moves proximally or distally, protrusion 160 may move along slot 154. In some aspects, protrusion(s) 160 may be slightly larger than slots 154 when slots 154 are in a natural, relaxed state. Thus, protrusion(s) 160 may provide resistance against distal movement of plunger 126. Material of plunger 126 may be sufficiently flexible that, upon application of a sufficient distal force (e.g., a distal force over a threshold value) to plunger 126, slots 154 slightly widen to allow distal movement of plunger 126 relative to housing 120.

During shipping and before actuation of actuator 122, actuator 122 may be “locked” in a fully retracted (fully proximal) position. As discussed above, O-ring 152 or ridge 150 may interact with inner surfaces of second wall portion 164 to resist distal movement (e.g., unintentional distal movement) of actuator 122. Similarly, an interaction between slots 154 and protrusion(s) 160 may also resist distal movement (e.g., unintentional distal movement) of actuator 122.

A user may apply force to grip 124 to supply a sufficient distal force to actuator 122 to overcome the friction between ridge 150 or O-ring 152 and second wall portion 164, thereby disengaging the locking mechanism. In some aspects, ridge 150 or O-ring 152 may deform radially inward or second wall portion 164 may deform radially outwardly. In other aspects, a frictional force may be overcome without any such deformation.

Upon application of sufficient distal force, slots 154 on plunger 126 may widen slightly so that slots 154 may move distally along protrusion 160. As plunger 126 moves proximally or distally, protrusion 160 may slide along the length of slots 154. Slots 154 may also maintain the alignment of plunger 126 within channel 142. The interaction between slot 154 and protrusion 160 may also constrain the range of motion of plunger 126, limiting extension or retraction of needle 112. In some aspects, slot 154 may include depth variations or surface features that interact with protrusion 160 to provide additional tactile feedback or intermediate securing positions during actuation. For example, slot 154 may include shallow protrusions or indentations at specific intervals that correspond to predetermined deployment depths of needle 112. As protrusion 160 encounters these indentations or protrusions, it may create a slight resistance or "click" sensation, allowing the user to gauge the deployment depth without relying solely on visual cues.

FIGS. 3A and 3F show details of how outer sheath 108 is coupled to handle 104. As shown particularly in FIG. 3F, a proximal end of outer sheath 108 may be radially surrounded by a connector 180. Alternatively, connector 180 may be coupled to a proximalmost end of outer sheath 108 and may extend proximally from the proximalmost end of outer sheath 108. Connector 180 may also include a ridged portion 182, which consists of circumferential ridges or protrusions on the exterior surface of connector 180. In addition, connector 180 may have an enlarged portion 184. Enlarged portion 182 may have an annular shape that provides an overall outer shape of a cylinder, disc, or other shape (e.g., cuboid or rectangular prism). Enlarged portion 182 may have a wider diameter/width than the rest of connector 180. Enlarged portion 184 may be positioned at the proximal end of connector 180, just proximal to ridged portion 182.

Housing 120 may include a channel 188 configured to receive outer sheath 108 and connector 180. Channel 188 may have ribs or ridges configured to support connector 180. Channel 188 may extend from the distal portion of housing 120. Housing 120 may also include a cavity 186 configured to receive enlarged portion 184. Cavity 186 is shaped to receive and secure enlarged portion 184 of connector 180. For example, channels in housing 120 that are proximal and distal of cavity 186 may be smaller than cavity 186 and enlarged portion 184, so that enlarged portion 184 is retained in cavity 186. In aspects, cavity 186 may have a shape that is keyed to enlarged portion 184. Thus, cavity 186 may help to fixedly secure outer sheath 108 to housing 120.

Housing 120 may also include channel 190 proximal to cavity 186. Channel 190 may be narrower than cavity 186 and may be configured to movably receive inner sheath 110. The narrower dimensions of channel 190 help to restrain inner sheath 110 within housing 120, preventing lateral movement and allowing axial movement as needed during actuation.

As shown in FIG. 3A, a strain relief 192 may be positioned over connector 180 to provide additional support and to additionally secure outer sheath 108 fixedly relative to housing 120. FIG. 3F shows handle 104 with strain relief 192 removed to show details of connector 180. Ridges of ridged portion 182 of connector 180 may facilitate fixation of strain relief 192 to connector 180. Ridged portion 182 may also enhance friction, helping to inhibit relative movement between connector 180 and housing 120. Strain relief 192 may extend into and terminate within channel 188 of housing 120. In some aspects, strain relief 192 may be attached to connector 180 using a heat-shrinking process, which helps form a tight and durable bond.

As mentioned above, medical device 100 may include connector 194, positioned at the proximal end of grip 124 as shown in FIG. 1. In some configurations, connector 194 may be a Luer connector, providing a connection point for various accessories or instruments. For example, connector 194 may help the attachment of a suction device or a stylet 196. As shown in FIG. 3A, stylet 196 may be insertable into the lumen of inner sheath 110, extending through connector 194, and into the lumen of inner sheath 110 and needle 112. Stylet 196 may include a nitinol or other metal wire along its length, which may be used by the user to push a tissue sample out of needle 112 after sample acquisition or may be used to provide rigidity to shaft 106 at various points of a procedure.

Stylet 196 may be removable from medical device 100, such that a user may attach a syringe or other aspiration device to connector 194 before or after stylet 196 is withdrawn. For example, connector 194 may be configured to be coupled with a suction device, enabling the application of negative pressure through the lumen of inner sheath 110 and needle 112, aiding in tissue sample collection.

In operation, the user may guide shaft 106 of medical device 100 to the target site within a subject's body. Initially, actuator 122 may be in a locked position, as described above. To unlock actuator 122, the user may apply a distal force to grip 124.

To deploy needle 112, the user may move actuator 122 distally. This action may cause plunger 126 to move within channel 142 of housing 120. As plunger 126 moves, inner sheath 110 and needle 112 may advance relative to outer sheath 108. The user may monitor the position of needle 112 using markings on plunger 126, which may correspond to specific deployment depths. When needle 112 reaches the desired position, the user may apply suction through connector 194 to collect a tissue sample. In aspects, the application of suction may be omitted. The suction may draw tissue into needle 112 and/or inner sheath 110.

After sample collection, the user may retract needle 112 by moving actuator 122 proximally. Plunger 126 may move within channel 142 until widened portion 128 contacts proximal surface 146, indicating full retraction of needle 112. To remove the collected sample, the user may withdraw medical device 100 from the subject's body. The user may then insert stylet 196 through connector 194 and use stylet 196 to push the sample out of needle 112 for analysis.

In some cases, the user may repeat this process multiple times to collect additional samples. Between sample collections, the user may clean needle 112 or replace components, as necessary. In some aspects, after completing the procedure, the user may engage the locking mechanism of medical device 100 so that needle 112 may remain in a retracted position for safe handling and storage of medical device 100.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
 a shaft having an inner sheath and an outer sheath; and
 a handle including:
 a housing defining a channel, wherein the housing is longitudinally fixedly
coupled to the outer sheath;
 an actuator having a proximal grip portion and a plunger extending distally from the grip portion, wherein a distalmost end of the plunger includes a widened portion that has a larger width than more proximal portions of the plunger, and wherein the plunger is fixedly coupled to the inner sheath; wherein the widened portion is longitudinally movable within the channel, and wherein a distance between a first surface of the housing and a second surface of the housing defines a maximum distance by which the actuator is movable relative to the housing and a maximum distance by which the inner sheath is movable relative to the outer sheath.

2. The medical device of claim 1, wherein a distal portion of the inner sheath is coupled to a needle.

3. The medical device of claim 2, wherein the needle and the inner sheath have a same inner diameter.

4. The medical device of claim 2, wherein the inner sheath is more flexible than the needle.

5. The medical device of claim 1, wherein a portion of the plunger between the grip portion and the widened portion is narrower than the grip portion and the widened portion.

6. The medical device of claim 1, wherein the housing includes a first wall portion, wherein the first wall portion includes the first surface.

7. The medical device of claim 6, wherein the housing includes a second wall portion, wherein the second wall portion includes the second surface.

8. The medical device of claim 7, wherein the second surface is a distalmost surface of the second wall portion.

9. The medical device of claim 7, wherein there is a gap between the first wall portion and the second wall portion.

10. The medical device of claim 1, wherein the plunger includes a ridge or an O-ring.

11. The medical device of claim 10, wherein the ridge or the O-ring is configured to inhibit distal movement of the plunger when the plunger is in a fully retracted configuration.

12. The medical device of claim 10, wherein the plunger includes a slot, wherein the housing has a protrusion received within the slot.

13. The medical device of claim 12, wherein the protrusion and the slot are configured to inhibit distal movement of the plunger when the plunger is in a fully retracted configuration.

14. The medical device of claim 1, wherein the proximal end of the outer sheath is covered by a connector.

15. The medical device of claim 14, wherein a ridged portion of the connector is covered by a strain relief.

16. A medical device, comprising:
 a shaft having an inner sheath, an outer sheath, and a needle fixedly coupled to a distal end of the inner sheath; and
 a handle including:
 a housing fixedly coupled to the outer sheath;
 an actuator having a proximal grip portion and a plunger extending distally from the grip portion, wherein a distalmost end of the plunger includes a widened portion that has a larger width than a portion of the plunger between the widened portion and the grip portion, and wherein the plunger is fixedly coupled to the inner sheath; wherein the widened portion is longitudinally movable within the housing to actuate the inner sheath relative to the outer sheath.

17. The medical device of claim 16, wherein the housing includes a first wall portion including a first surface, and a second wall portion having a second surface, wherein the second surface is a distalmost surface of the second wall portion, wherein the first surface is a distal bound on movement of the widened portion, and wherein the second surface is a proximal bound on movement of the widened portion.

18. A medical method, comprising:
advancing a shaft of a medical device to a target site within a subject’s body, wherein the shaft includes an outer sheath and an inner sheath, and wherein the medical device includes a handle with a housing and an actuator, wherein the actuator includes a plunger having a widened portion coupled to the inner sheath;
applying a distal force to the actuator to unlock a locking mechanism; and
moving the actuator distally relative to the housing, wherein the widened portion moves distally within the housing to move the inner sheath distally relative to the outer sheath, wherein a stroke length of the actuator is defined by a distance between a first surface and a second surface of the housing, wherein the widened portion abuts the first surface in a fully retracted configuration of the inner sheath and abuts the second surface in a fully extended configuration of the inner sheath.

19. The medical method of claim 18, wherein the locking mechanism includes a ridge or an O-ring on the plunger.

20. The medical method of claim 18, wherein the locking mechanism includes at least one longitudinal slot on the plunger.
